(19) United States
(12) Patent Application Publication
(10) Pub. No.: US 2011/0166223 A1
(43) Pub. Date: Jul. 7, 2011

(54) METHODS OF INHIBITING FGFR3 SIGNALING

(75) Inventors: Pavel Krejci, Los Angeles, CA (US); William Wilcox, Los Angeles, CA (US)

(73) Assignee: CEDARS-SINAI MEDICAL CENTER, Los Angeles, CA (US)

(21) Appl. No.: 13/057,975

(22) PCT Filed: Aug. 19, 2009
(86) PCT No.: PCT/US2009/054340

§ 371 Date: Feb. 7, 2011

Related U.S. Application Data
(60) Provisional application No. 61/090,168, filed on Aug. 19, 2008.

(51) Int. Cl. A61K 31/255; A61P 19/00; A61P 35/00; A61P 13/10

(52) U.S. Cl. 514/518

(57) ABSTRACT

Novel inhibitors of FGFR3 signaling having a structure shown as Formula 1 and a method of inhibiting FGFR3 signaling by administering a quantity of the inhibitor, or pharmaceutical equivalent, analog and/or salt thereof, to a mammal are disclosed. Additionally, the inhibitor may be used for treating one or more conditions associated with FGFR3 mediated signaling

### FIELD OF INVENTION

The invention relates to the fields of molecular biology and medicine. More specifically, the invention relates to compounds and methods capable of modulating cell signaling mediated by FGFR3.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Fibroblast growth factor receptor 3 (FGFR3) is a transmembrane tyrosine kinase that serves as a receptor for the members of fibroblast growth factor (FGF) family, and functions in many biological processes including cell proliferation, differentiation, migration and survival. Activating mutations in FGFR3 were found associated with several human disorders such as skeletal dysplasias, multiple myeloma (MM), and cervical and bladder carcinomas [1-4]. Among skeletal dysplasias, activating FGFR3 mutations cause achondroplasia, the most common form of human skeletal dysplasia, and thanatophoric dysplasia, the most common form of lethal skeletal dysplasia [1]. Long bones of individuals suffering from FGFR3-related skeletal dysplasias show markedly shortened zones of chondrocyte proliferation and differentiation, while Fgfr3 knockout mice demonstrate skeletal overgrowth, together implying the role of FGFR3 as a negative regulator of bone growth [5].

Apart from cartilage, approximately 15% of patients suffering from multiple myeloma (MM) markedly upregulate FGFR3 as a consequence of a t(4;14)(p16.3;q32) translocation, with a fraction of patients also harboring activating mutations in FGFR3, similar to those found in skeletal dysplasias [2, 3]. Ectopic expression of FGFR3 enhances MM cell proliferation and survival, thus demonstrating the oncogenic potential of FGFR3 [6].

Given its role in human disease, FGFR3 signaling represents an attractive target for therapy. Thus, there is a need in the art for novel inhibitors of FGFR3 signaling as well as the development of effective treatments for FGFR3 mediated diseases and conditions.

### SUMMARY OF THE INVENTION

Various embodiments include a method of inhibiting fibroblast growth factor 2 (FGF2) and/or fibroblast growth factor receptor 3 (FGFR3) mediated signaling in a mammal, comprising providing a quantity of composition comprising a compound the formula:

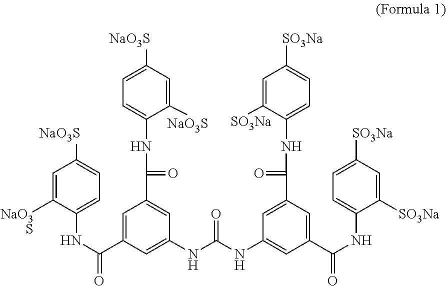

or a pharmaceutical equivalent, analog and/or salt thereof, and administering the quantity of the composition to the mammal. In another embodiment, the composition comprises 5 to 30 μM of the compound of Formula 1 or the pharmaceutical equivalent, analog and/or salt thereof. In another embodiment, the composition comprises greater than 0.1 μM of the compound of Formula 1 or the pharmaceutical equivalent, analog and/or salt thereof. In another embodiment, the composition comprises at least 2 μM of the compound of Formula 1 or the pharmaceutical equivalent, analog and/or salt thereof. In another embodiment, the composition comprises 25 μM of the compound of Formula 1 or the pharmaceutical equivalent, analog and/or salt thereof. In another embodiment, inhibiting FGF2 and/or FGFR3 mediated signaling results in rescue of growth arrest and/or extracellular matrix loss.

Other embodiments include a method of treating an FGF2 and/or FGFR3 mediated disorder in a subject, comprising providing a quantity of a composition comprising a compound of the formula:

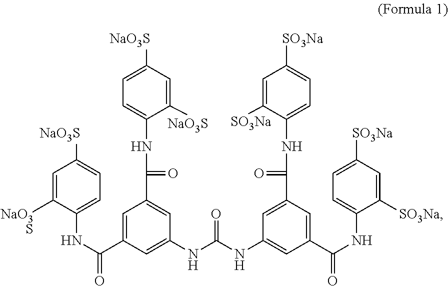

or a pharmaceutical equivalent, analog and/or salt thereof, and administering the quantity of the composition to the subject. In another embodiment, the FGF2 and/or FGFR3 mediated disorder comprises a skeletal disorder, skeletal dysplasia, multiple myeloma, cervical carcinoma, and/or bladder carcinoma. In another embodiment, the composition comprises 25 μM of the compound of Formula 1, or the pharmaceutical equivalent, analog and/or salt thereof. In another embodiment, the composition comprises from 1 μM to 30 μM of the compound of Formula 1, or the pharmaceutical equivalent, analog and/or salt thereof. In another embodiment, the composition is administered to the subject intravenously. In another embodiment, the composition is administered to the subject by direct injection. In another embodiment, FGF2 and/or FGFR3 mediated signaling is inhibited by direct inhibition of FGFR3 kinase activity.

Other embodiments include a pharmaceutical composition, comprising a therapeutically effective amount of a compound of the formula:

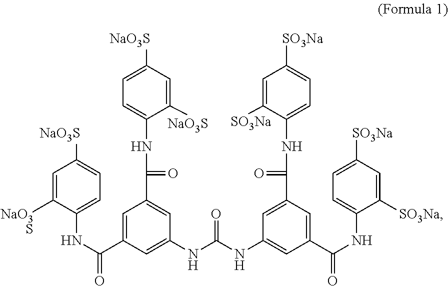

or a pharmaceutical equivalent, analog and/or salt thereof, and a pharmaceutically acceptable carrier.

Various embodiments include a method of treating an FGF2 and/or FGFR3 mediated condition in a subject, comprising administering a quantity of a composition comprising a compound of the formula:

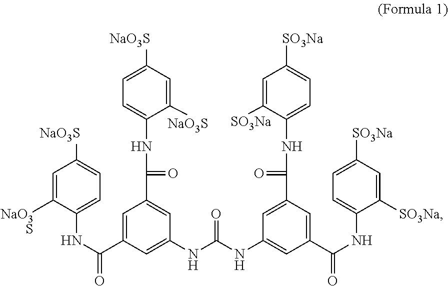

or a pharmaceutical equivalent, analog and/or salt thereof, to the subject, and administering a quantity of a composition comprising a C-natriuretic peptide (CNP) compound, or a pharmaceutical equivalent, analog, derivative and/or salt thereof, to the subject.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1, in accordance with an embodiment herein, depicts 4,4′,4″,4′″-[carbonyl-bis[imino-5,1,3-benzenetriyl bis-{carbonylimino}]]tetrakis-{benzene-1,3-disulfonic acid} inhibits FGF2-mediated growth arrest in chondrocytes. (a) RCS chondrocytes were treated with 5 ng/ml of FGF2 and various concentrations of 4,4′,4″,4′″-[carbonyl-bis[imino-5,1,3-benzenetriyl bis-{carbonylimino}]]tetrakis-{benzene-1,3-disulfonic acid} (upper graph), or with 15 μM 4,4′,4″,4′″-[carbonyl-bis[imino-5,1,3-benzenetriyl bis-{carbonylimino}]]tetrakis-{benzene-1,3-disulfonic acid} and various FGF2 concentrations (lower graph; samples containing no FGF2 were given an artificial value of 0.8 on the logarithmic x-axis) for 72 hours and counted. Data represent an average from four wells with the indicated standard deviation. Note the potent growth arrest induced by FGF2 as well as the 4,4′,4″,4′″-[carbonyl-bis[imino-5,1,3-benzenetriyl bis-{carbonylimino}]]tetrakis-{benzene-1,3-disulfonic acid}-mediated reversal of this phenotype. Also note the lack of significant 4,4′,4″,4′″-[carbonyl-bis[imino-5,1,3-benzenetriyl bis-{carbonylimino}]]tetrakis-{benzene-1,3-disulfonic acid} toxicity throughout its active range (8-30 μM; upper graph). (b) RCS chondrocytes were treated as indicated, grown for 72 hours and counted. Note the cumulative effect of both CNP and 4,4′,4″,4′″-[carbonyl-bis[imino-5,1,3-benzenetriyl bis-{carbonylimino}]]tetrakis-{benzene-1,3-disulfonic acid} on the FGF2-mediated growth arrest. Data represent an average from four wells with the indicated standard deviation. Statistical differences are indicated (Student's t-test; *-p<0.01).

FIG. 2, in accordance with an embodiment herein, depicts 4,4′,4″,4′″-[carbonyl-bis[imino-5,1,3-benzenetriyl bis-{carbonylimino}]]tetrakis-{benzene-1,3-disulfonic acid} inhibits FGF2-mediated extracellular matrix degradation in chondrocytes. (a) RCS chondrocytes were treated with FGF2 (5 ng/ml) alone or in the presence of 4,4′,4″,4′″-[carbonyl-bis[imino-5,1,3-benzenetriyl bis-{carbonylimino}]]tetrakis-{benzene-1,3-disulfonic acid} (20 μM) for 72 hours and the cell extracellular matrix was visualized by alcian blue staining (left panel; 200×). The corresponding dark field photograph is also shown (right panel). Note the FGF2-mediated extracellular matrix loss was reversed by 4,4′,4″,4′″-[carbonyl-bis[imino-5,1,3-benzenetriyl bis-{carbonylimino}]]tetrakis-{benzene-1,3-disulfonic acid}. Also note the marked cellular shape change induced by FGF2 that was also rescued by 4,4′,4″,4′″-[carbonyl-bis[imino-5,1,3-benzenetriyl bis-{carbonylimino}]]tetrakis-{benzene-1,3-disulfonic acid}. (b) RCS chondrocytes were treated as indicated in the presence of [³⁵S]sulfate for 72 hours, and the amount of incorporated radioactivity was determined as described herein. Note the FGF2-mediated loss of sulfated proteoglycans was reversed by 4,4′,4″,4′″-[carbonyl-bis[imino-5,1,3-benzenetriyl bis-{carbonylimino}]]tetrakis-{benzene-1,3-disulfonic acid}. Data are average from four wells with indicated standard deviation. Statistically significant differences are indicated (Student's t-test; *-p<0.01). Results are representative of three experiments.

FIG. 3, in accordance with an embodiment herein, depicts 4,4′,4″,4′″-[carbonyl-bis[imino-5,1,3-benzenetriyl bis-{carbonylimino}]]tetrakis-{benzene-1,3-disulfonic acid} inhibits FGF2/FGFR3 signaling in MM cell lines OPM2 and KMS11. (a) KMS11 and OPM2 cells were serum-starved for 12 hours, treated with 4,4′,4″,4′″-[carbonyl-bis[imino-5,1,3-benzenetriyl bis-{carbonylimino}]]tetrakis-{benzene-1,3-disulfonic acid} for one hour before the treatment with FGF2 and the levels of ERK MAP kinase phosphorylation were determined by western blotting with the P-ERK1/2(Thr²⁰²/Tyr²⁰⁴) antibody. As a loading control, the membranes were reprobed with antibody recognizing ERK regardless of its phosphorylation. Note the FGF2-mediated ERK phosphorylation is inhibited by 4,4′,4″,4′″-[carbonyl-bis[imino-5,1,3-benzenetriyl bis-{carbonylimino}]]tetrakis-{benzene-1,3-disulfonic acid} in both KMS11 and OPM2 cells. (b) The OPM2 cells were serum starved for 12 hours, pre-treated with 4,4′,4″,4′″-[carbonyl-bis[imino-5,1,3-benzenetriyl bis-{carbonylimino}]]tetrakis-{benzene-1,3-disulfonic acid} for one hour before FGF2 treatment, and analyzed for CCL3 and CCL4 expression by real-time RT-PCR as described herein. The results are expressed as the fold difference relative to control (2^{ddC}ₜ) [9]. Note the FGF2-mediated induction of CCL3 and CCL4 that is inhibited by 4,4′,4″,4′″-[carbonyl-bis[imino-5,1,3-benzenetriyl bis-{carbonylimino}]]tetrakis-{benzene-1,3-disulfonic acid}. The data represent two independent samples, each run as a technical duplicate, with the indicated range. The results are representative for three experiments.

FIG. 4, in accordance with an embodiment herein, depicts 4,4′,4″,4′″-[carbonyl-bis[imino-5,1,3-benzenetriyl bis-{carbonylimino}]]tetrakis-{benzene-1,3-disulfonic acid} inhibits FGFR3 activity in a kinase assay. (a, b) Cell-free kinase assays were carried out as described herein, with recombinant tyrosine kinase (TK) domain of FGFR3 as a kinase, recombinant STAT1 as a substrate, and NF449 added directly to the kinase reaction. FGFR3-mediated phosphorylation of STAT1 was detected by WB with P-STAT1 (Y⁷⁰¹) antibody. The membrane was reprobed with FGFR3 and STAT1 antibodies to control for kinase and substrate quantity. The sample with ATP omitted serves as a negative control for the kinase reaction. Note the FGFR3-mediated phosphorylation of STAT1 is inhibited by 4,4′,4″,4′″-[carbonyl-bis[imino-5,1,3-benzenetriyl bis-{carbonylimino}]]tetrakis-{benzene-1,3-disulfonic acid}. The P-STAT1 and STAT1 signal was quantified by densitometry and expressed as a ratio between P-STAT1 and STAT1 signal for each given sample (b, lower graph). (c) The FGFR3 kinase assay was carried-out as described in (a) with 4,4′,4″,4′″-[carbonyl-bis[imino-5,1,3-benzenetriyl bis-{carbonylimino}]]tetrakis-{benzene-1,3-disulfonic acid}, 8,8′-[carbonylbis[imino-3,1-phenylenecarbonylimino{4-methyl-3,1-phenylene}carbonylimino]]bis-1,3,5-naphthalenetrisulfonic acid or 8,8′-[carbonylbis[imino-3,1-phenylene]]bis-{1,3,5-naphthalenetrisulfonic acid} added to the kinase reaction. Note the differential effects of 8,8′-[carbonylbis[imino-3,1-phenylenecarbonylimino{4-methyl-3,1-phenylene}carbonylimino]]bis-1,3,5-naphthalenetrisulfonic acid and 8,8′-[carbonylbis[imino-3,1-phenylene]]Bis-{1,3,5-naphthalenetrisulfonic acid} on the FGFR3-mediated phosphorylation of STAT1, which corresponds to their activity in the RCS growth-arrest experiment (lower graph). Data are an average from four wells with the indicated standard deviation. Statistically significant rescue of the growth arrest is indicated (Student's I-test; *-p<0.01).

FIG. 5, in accordance with an embodiment herein, depicts chemical structures of: (a) 8,8′-[carbonylbis[imino-3,1-phenylenecarbonylimino {4-methyl-3,1-phenylene}carbonylimino]]bis-1,3,5-naphthalenetrisulfonic acid; (b) 8,8′-[carbonylbis[imino-3,1-phenylene]]bis-{1,3,5-naphthalenetrisulfonic acid}; and (c) 4,4′,4″,4′″-[carbonyl-bis[imino-5,1,3-benzenetriyl bis-{carbonylimino}]]tetrakis-{benzene-1,3-disulfonic acid}.

FIG. 6, in accordance with an embodiment herein, depicts 4,4′,4″,4′″-[carbonyl-bis[imino-5,1,3-benzenetriyl bis-{carbonylimino}]]tetrakis-{benzene-1,3-disulfonic acid} inhibits K650E-FGFR3 in a kinase assay. Cell-free K650E-FGFR3 kinase assays were carried-out using full-length FLAG-tagged K650E-FGFR3 that was expressed in CHO cells, purified by immunoprecipitation with FLAG antibody and subjected to a kinase reaction with recombinant STAT1 as a substrate and 4,4′,4″,4′″-[carbonyl-bis[imino-5,1,3-benzenetriyl bis-{carbonylimino}]]tetrakis-{benzene-1,3-disulfonic acid} added to the kinase reaction. Cells transfected with plasmid encoding green fluorescent protein (GFP) serve as a negative control for the immunoprecipitation. Note the inhibitory effect of NF449 on the FGFR3-mediated Y⁷⁰¹ phosphorylation of STAT1.

FIG. 7, in accordance with an embodiment herein, depicts a chart demonstrating the effect of administering FGFR3 and 4,4′,4″,4′″-[carbonyl-bis[imino-5,1,3-benzenetriyl bis-{carbonylimino}]]tetrakis-{benzene-1,3-disulfonic acid} on limb size.

FIG. 8, in accordance with an embodiment herein, depicts embryos cultured for six (6) days. 30 μM 4,4′,4″,4′″-[carbonyl-bis[imino-5,1,3-benzenetriyl bis-{carbonylimino}]]tetrakis-{benzene-1,3-disulfonic acid} administered resulted in greater limb growth and size.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, “FGFR3” means fibroblast growth factor receptor 3.

As used herein, “FGF” means fibroblast growth factor.

As used herein, “RCS” means rat chondrosarcoma.

As used herein, “MM” means multiple myeloma.

As used herein, “NF449 composition” means a composition including NF449, or an analog, salt or pharmaceutical equivalent thereof.

As used herein, the term “NF449” means Formula 1 and also includes the following: 4,4′,4″,4′″-[carbonylbis(imino-5,1,3-benzenetriyl-bis(carbonylimino))]tetrakis-1,3-benzenedisulfonic acid, octasodium salt; 4,4′,4″,4′″-[carbonyl-bis[imino-5,1,3-benzenetriyl bis-(carbonyl-imino)]]tetrakis(benzene-1,3-disulfonic acid) octasodium salt; 4,4′,4″,4′″-[carbonyl-bis[imino-5,1,3-benzenetriyl bis-(carbonylimino)]]tetrakis-(benzene-1,3-disulfonic acid, 8NA); NF449 octasodium salt; 4,4′,4″,4′″-(Carbonylbis(imino-5,1,3-benzenetriyl-bis(carbonylimino)))tetrakis-1,3-benzenedisulfonic acid, octasodium; 4,4μ,4,4μ-[Carbonylbis[imino-5,1,3-benzenetriylbis(carbonyl-imino)]]tetrakis(benzene-1,3-disulfonic acid) octasodium salt; 4,4′,4″,4′″-[Carbonylbis(imino-5,1,3-benzenetriyl-bis(carbonylimino))]tetrakis-1,3-benzenedisulfonic acid octasodium salt.

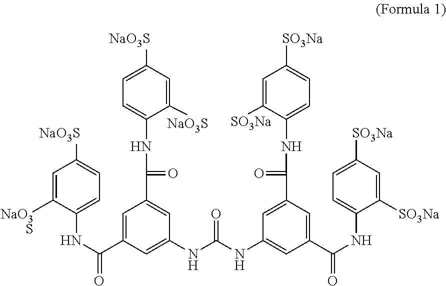

As disclosed herein, using a molecular library screening approach, a compound named NF449 was identified based on its inhibitory activity towards FGFR3 signaling. FGFR3 receptor tyrosine kinase represents an attractive target for therapy due to its candidate role in several human disorders such as skeletal dysplasias, multiple myeloma, and cervical and bladder carcinomas.

As further disclosed herein, the Formula 1 compound (also known as NF449), is an example of a benzenesulfonate, or salt or ester of a benzenesulfonic acid, or besylate. Benzenesulfonates, in turn, may be classified as a type of benzene derivative or arylsulfonate.

As further disclosed herein, the Formula 1 compound rescued both major phenotypes of pathological. FGFR3 signaling in skeletal dysplasias, i.e. FGFR3-mediated chondrocyte growth arrest and extracellular matrix loss. Similarly, the Formula 1 compound antagonized FGFR3 signaling in the multiple myeloma cell lines OPM2 and KMS11, as evidenced by Formula 1-mediated reversal of ERK MAP kinase activation and transcript accumulation of CCL3 and CCL4 chemokines, both of which are induced by FGFR3 activation. In cell-free kinase assays utilizing STAT1 as a substrate, Formula 1 compound inhibited the tyrosine kinase activity of recombinant FGFR3 as well as immunopurified K650E-FGFR3, which represents a highly activated FGFR3 mutant associated with both skeletal dysplasia and multiple myeloma. Taken together, the data identifies Formula 1 as an antagonist of FGFR3 tyrosine kinase, active in chondrocytes and multiple myeloma cells.

In one embodiment, the present invention provides a method of treating a disease and/or condition in a mammal by administering a therapeutically effective amount of a composition comprising a benzenesulfonate compound, or pharmaceutical equivalent, analog and/or salt thereof. In another embodiment, the benzenesulfonate compound is Formula 1, or pharmaceutical equivalent, analog, and/or salt thereof. In another embodiment, the disease and/or condition is a growth defect. In another embodiment, the disease and/or condition is achondroplasia. In another embodiment, the disease and/or condition is cancer. In another embodiment, the disease and/or condition is multiple myeloma. In another embodiment, the disease and/or condition is oncogenic FGFR3 signaling. In another embodiment, the disease and/or condition is RCS proliferation. In another embodiment, the disease and/or condition is skeletal dysplasias, multiple myeloma, and cervical and bladder carcinomas. In another embodiment, the mammal is a rat. In another embodiment, the mammal is a human.

In one embodiment, the present invention provides a method of inhibiting FGFR3 mediated signaling in a cell by administering an effective dosage of Formula 1, or pharmaceutical equivalent, analog, and/or salt thereof. In another embodiment, the cell is KMS11, OPM2 and/or RCS.

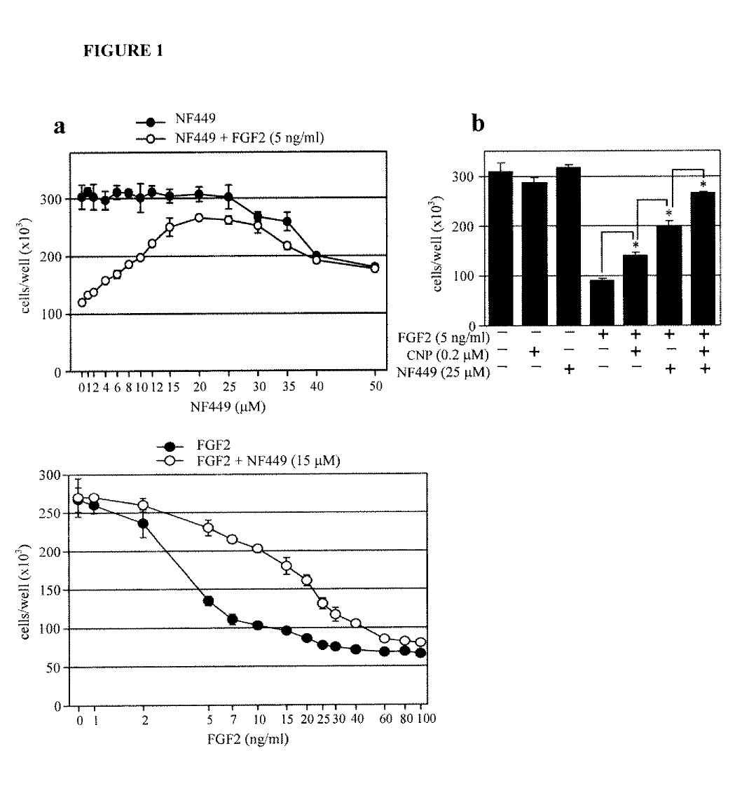

As disclosed herein, the inventors also compared the Formula 1 compound effect with that of C-natriuretic peptide (CNP). CNP is recently discovered, potentially therapeutic antagonist of FGFR3 signaling that suppresses the pathological FGFR3 signaling in cartilage via inhibition of one of its intermediates, the ERK MAP kinase pathway [12, 13]. FIG. 1b shows that CNP caused ˜23% rescue of the FGF2-mediated RCS growth-arrest, similar to previous data [1,2]. This effect was significantly exceeded by Formula 1 compound which lead to ˜50% rescue of the growth arrest phenotype. When used together, CNP and Formula 1 compound acted cumulatively, rescuing nearly 80% of the growth arrest.

In one embodiment, the present invention provides a method of treating an FGF2-mediated condition by administering a first therapeutically effective amount of a composition comprising a Formula 1 compound, or pharmaceutical equivalent, analog, and/or salt thereof, and administering a second therapeutically effective amount of a composition comprising a CNP compound, or pharmaceutical equivalent, analog and/or salt thereof, wherein the first therapeutically effective amount of a composition comprising a Formula 1 compound, or pharmaceutical equivalent, analog, and/or salt thereof, and administering a second therapeutically effective amount of a composition comprising a CNP compound, or pharmaceutical equivalent, analog, derivative and/or salt thereof, have a cumulative effect on ameliorating the FGF2-mediated condition.

The present invention is also directed to a kit to prepare a composition comprising a Formula 1 compound, or pharmaceutical equivalent, analog, and/or salt thereof, as well as the delivery of the composition comprising the Formula 1 compound, or pharmaceutical equivalent, analog, and/or salt thereof to an individual, and may include serum, cells, probes, reporter constructs, antibodies, kinase assays, and combinations thereof. The kit is an assemblage of materials or components, including at least one of the inventive compositions.

Instructions for use may be included in the kit. “Instructions for use” typically include a tangible expression describing the technique to be employed in using the components of the kit to effect a desired outcome, such as to prepare a composition comprising a Formula 1 compound, or pharmaceutical equivalent, analog, and/or salt thereof, and/or deliver a therapeutically effective dosage of composition comprising a Formula 1 compound, or pharmaceutical equivalent, analog, and/or salt thereof to treat a disease mediated by FGFR3 signaling. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing a solution comprising a Formula 1 compound, or pharmaceutical equivalent, analog, and/or salt thereof, or components thereof. The packaging material generally has an external label which indicates the contents and/or purpose of the kit and/or its components.

In various embodiments, the present invention provides pharmaceutical compositions including a pharmaceutically acceptable excipient along with a therapeutically effective amount of Formula 1 compound, or pharmaceutical equivalent, analog, and/or salt thereof. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to an osmotic minipump, intravenous injection, aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The composition comprising a Formula 1 compound, or pharmaceutical equivalent, analog, and/or salt thereof according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The composition comprising a Formula 1 compound, or pharmaceutical equivalent, analog, and/or salt thereof according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The preparations of a composition comprising a Formula 1 compound, or pharmaceutical equivalent, analog, and/or salt thereof are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The composition comprising a Formula 1 compound, or pharmaceutical equivalent, analog, and/or salt thereof according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of a composition comprising a Formula 1 compound, or pharmaceutical equivalent, analog, and/or salt thereof can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as the responses observed in the appropriate animal models, as previously described.

As readily apparent to one of skill in the art, a condition or disorder that is mediated by FGFR3 and/or FGF2 signaling may be treated by effectively inhibiting or down regulating FGFR3 and/or FGF2 signaling mechanism, thereby ameliorating one or more conditions that would otherwise be associated with the FGFR3 and/or FGF2 signaling. For example, FGFR3 is a major negative regulator of growth in children, so that inhibition of FGFR3 could treat short stature of the subject, such as the condition of dwarfism or skeletal dysplasias. As readily apparent to one of skill in the art, the invention may be applied to any number of FGFR3 mediated conditions and diseases, including seborrheic keratosis, cancer, multiple myeloma, achondroplasia and dwarfism.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### NF449 Inhibition of FGFR3Signaling

The inventors developed a mammalian cell-based screening assay suitable for identification of inhibitors of FGFR3 signaling (Krejci, et al., Invest New Drugs 2007, 25: 391-395). In this assay, the FGF2/FGFR3-mediated growth-arrest of rat chondrosarcoma (RCS) cells is used as a reporter, thus allowing for elimination of toxic compounds as false-positive hits (Krejci, et al., Invest New Drugs 2007, 25: 391-395). The inventors used the RCS growth-arrest assay to screen a library consisting of 1120 molecules (Tocris Bioscience, Ellisville, Mo.) for chemicals active against FGFR3 signaling. Screening performed at the 20 μM scale identified a compound named NF449 as an antagonist of the FGF2/FGFR3 inhibitory effect on RCS proliferation. This effect was confirmed in detailed cell-growth experiments, where 25 μM NF449 caused nearly 70% rescue of the growth-arrest phenotype without significant cell toxicity throughout its active concentration range (8-30 μM). This activity of NF449 is especially remarkable considering the robustness of the RCS growth-arrest phenotype (Krejci, et al., Invest New Drugs 2007, 25: 391-395).

The inventors then determined whether NF449 also inhibits FGFR3 signaling in KMS11 and OPM2 cell lines, which serve as a model for oncogenic FGFR3 signaling in multiple myeloma (Ronchetti, et al., Oncogene 2001; 20: 3553-3562). As a surrogate for FGFR3 activation, the inventors used ERK MAP kinase, which is activated by addition of exogenous FGF2 to the OPM2 or KMS11 cells (Krejci, et al., Leukemia 2006; 20: 1165-1.168. As disclosed herein, FGF2 stimulation leads to the activatory Thr²⁸²/Tyr²⁰⁴ phosphorylation of ERK, which was almost completely blocked by NF449. To further confirm this data, the inventors analyzed the NF449 effect on the genes induced by FGFR3 signaling in MM cells. As the CCL3 (MIP-1α) and CCL4 (MIP-1β) chemokines were previously identified as transcriptional targets of FGFR3 signaling in multiple myeloma (Masih-Khan, et al., Blood 2006; 108: 3465-3471), the inventors determined the effect of NF449 on the FGF2/FGFR3-induced expression of CCL3 and CCL4. Real-time RT-PCR analysis showed that FGF2-treatment of OPM2 cells leads to significant upregulation of transcripts for both CCL3 and CCL4 that was significantly blocked by NF449.

NF449 was originally described as a G protein antagonist selective to the G_{SA} subunit (Hohenegger, et al., Proc Natl Acad Sci USA 1998; 95: 346-351). Although NF449 activity against the FGFR3 signaling opens the possibility of involvement of the G proteins, the NF449 concentrations active in the RCS growth-arrest assay were higher than those needed to inhibit G_{SA} (8-30 μM versus 0.14 M) (Hohenegger, et al., Proc Natl Acad Sci USA 1998; 95: 346-351), thus suggesting another target for NF449. The inventors therefore tested the NF449 effect on FGFR3 kinase activity in a cell-free FGFR3 kinase assay that employs recombinant tyrosine kinase domain of FGFR3 as a kinase and STAT1 as a substrate (Krejci, et al., J Cell Sci 2008; 121: 272-281). In this experiment, NF449 inhibited the FGFR3-mediated tyrosine phosphorylation of STAT1. The inventors next tested two other G-protein inhibitors structurally similar to NF449, i.e. suramin and NF023, for their activity against FGFR3. Suramin inhibited FGFR3 kinase activity in contrast to NF023, which had no effect. These data corresponds to the activity of suramin and NF023 in the RCS growth-arrest assay, where suramin reversed the FGF2/FGFR3-mediated growth arrest in a manner similar to NF449 in contrast to NF023 which had no effect.

Since OPM2 cells express FGFR3 with a highly activating K650E mutation (Ronchetti, et al., Oncogene 2001; 20: 3553-3562), the inventors used the cell-free kinase assay to determine to what extent the K650E-FGFR3 is inhibited by NF449. As described in detail elsewhere (Krejci, et al., J Cell Sci 2008; 121: 272-281), FLAG-tagged full-length FGFR3-K650E was expressed in CHO cells, purified by immunoprecipitation 48 hours later and subjected to a kinase assay with STAT1 as a substrate and NF449 added directly to the kinase reaction.

Taken together, the inventors have identified the NF449 as a novel, water-soluble and non-toxic inhibitor of FGFR3 signaling with therapeutic application in multiple myeloma and FGFR3-related skeletal dysplasias. The inventors also show that NF449 could inhibit FGFR3 signaling by directly targeting FGFR3 kinase activity.

### Example 2

### Generally

FGFR3 receptor tyrosine kinase represents an attractive target for therapy due to its candidate role in several human disorders such as skeletal dysplasias, multiple myeloma, and cervical and bladder carcinomas. Using a molecular library screening approach, the inventors identified a compound named NF449 based on its inhibitory activity towards FGFR3 signaling. NF449 rescued both major phenotypes of pathological FGFR3 signaling in skeletal dysplasias, i.e. FGFR3-mediated chondrocyte growth arrest and extracellular matrix loss. Similarly, NF449 antagonized FGFR3 signaling in the multiple myeloma cell lines OPM2 and KMS11, as evidenced by NF449-mediated reversal of ERK MAP kinase activation and transcript accumulation of CCL3 and CCL4 chemokines, both of which are induced by FGFR3 activation. In cell-free kinase assays utilizing STAT1 as a substrate, NF449 inhibited the tyrosine kinase activity of recombinant FGFR3 as well as immunopurified K650E-FGFR3, which represents a highly activated FGFR3 mutant associated with both skeletal dysplasia and multiple myeloma. Taken together, the data identifies NF449 as a novel antagonist of FGFR3 tyrosine kinase, active in chondrocytes and multiple myeloma cells.

### Example 3

### Cell Culture and Growth Assays

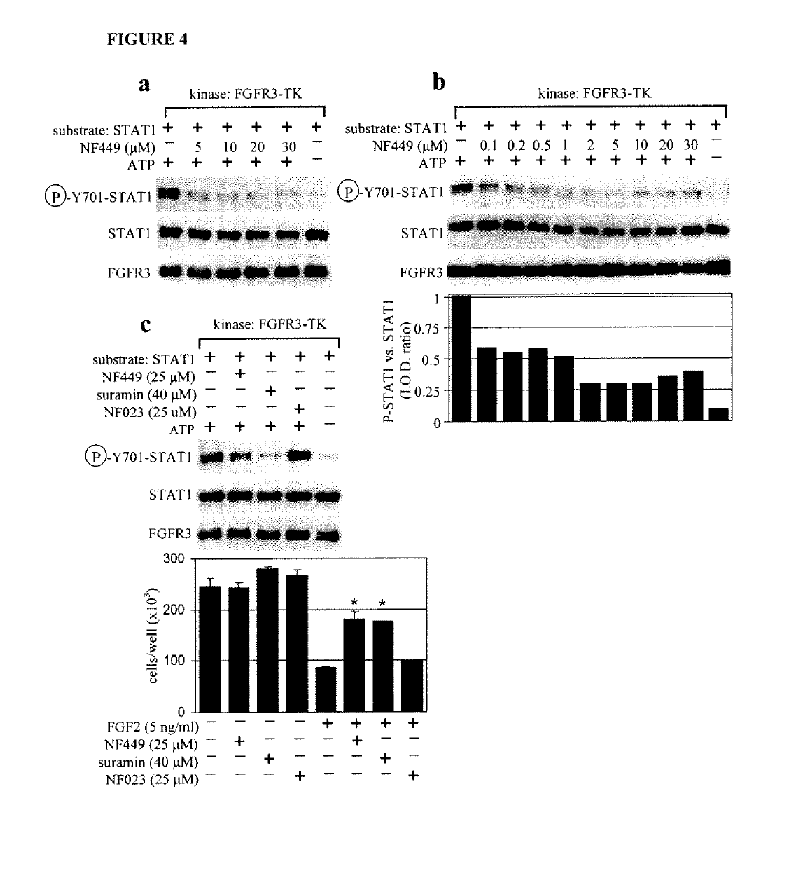

Rat chondrosarcoma chondrocytes (RCS), chinese hamster ovary (CHO) cells and MM cell lines OPM2 and KMS11 were propagated in DMEM, Opti-MEM or RPMI media (Gibco, Gaitherbsburg, Md.), supplemented with 10% fetal bovine serum (Atlanta Biological, Nordeross, Ga.) and antibiotics. The RCS growth experiments utilizing crystal violet staining to quantify cell growth are described in detail elsewhere [7]. For the RCS growth-arrest experiments shown at FIGS. 1 and 4c, 1×10⁴ RCS chondrocytes were seeded in 24-well tissue culture plates (Costar, Cambridge, Mass.), treated as indicated for 72 hours, and counted. FGF2 was obtained from R&D Systems (Minneapolis, Minn.); C-natriuretic peptide, suramin, NF023 and NF449 were from Calbiochem (San Diego, Calif.).

### Example 4

### Alcian Blue Staining and [³⁵S]Sulfate Labeling Assays

For Alcian blue staining, growing RCS cultures were treated with FGF2 (5 ng/ml) alone or in the presence of NF449 (20 μM) for 72 hours, fixed with 4% paraformaldehyde and stained for 30 minutes with Alcian blue. For quantification of the FGF2/FGFR3-mediated extracellular matrix loss, RCS chondrocytes were treated with FGF2 (5 ng/ml) and NF449 (20 μM) for 72 hours in the presence of 10 μCi/ml of [³⁵S]sulfate (Perkin Elmer, Boston, Mass.). Following the cultivation period, cells were harvested and the incorporated radioactivity was determined by liquid scintillation.

### Example 5

### Preparation of Cell Extracts, Western Blotting, Immunoprecipitation and Kinase Assays

OPM2 and KMS11 cells were lysed in ice-cold immunoprecipitation buffer (50 mM Tris-HCl pH 7.4, 150 mM NaCl, 0.5% NP-40, 1 mM EDTA, 25 mM NaF) supplemented with proteinase inhibitors and 10 mM sodium orthovanadate. Protein samples were resolved by SDS-PAGE and transferred onto a PVDF membrane. The following antibodies were used: Erk1/2, P-Erk1/2^{T202/Y204}, STAT1, P-STAT1^{Y701} (Cell Signaling, Beverly, Mass.) and FGFR3 (Santa Cruz Biotechnology, Santa Cruz, Calif.). Western blotting signal was quantified by determining the integrated optical density (I.O.D.) of a given band using Scion Image software (Scion Corporation, Frederick, Mass.). Immunoprecipitations and FGFR3 kinase assays were performed as described [8]. Briefly, the pRK7 vector encoding C-terminally FLAG-tagged K650E-FGFR3 was transfected into CHO cells using FuGENE6 reagent according to the manufacturer's protocol (Roche Applied Science, Indianapolis, Ind.). 48 hours after transfection, cells were treated with 20 ng/ml of FGF2 for 15 minutes and K650E-FGFR3 was immunoprecipitated from 800 μg of cell lysate protein using 4 μg of anti-FLAG antibody (Sigma-Aldrich, St. Louis, Mo.). Cells transfected with a plasmid encoding green fluorescent protein (pCCEY) serve as a negative control for the immunoprecipitation. Immunocomplexes were washed two times with kinase buffer (60 mM HEPES-NaOH pH 7.5, 3 mM MgCl₂, 3 mM MnCl₂, 3 μM Na₃VO₄, 1.2 mM dithiothreitol), and the kinase reactions were performed for 30 minutes at 30° C. in the presence of 2.5 μg of polyethylene glycol, 10 μM ATP, 300 ng of recombinant STAT1 (Active Motif, Carlsbad, Calif.) as a substrate, and NF449 added directly to the kinase reaction. Kinase assays utilizing recombinant tyrosine kinase (TK) domain of FGFR3 (Glu-322-Thr-806; Cell Signaling) were carried-out similarly, with 300 ng of kinase used in 50 μl reaction, and suramin, NF449 and NF023 added directly to the kinase reaction. FGFR3-mediated phosphorylation of STAT1 was detected by western blotting with P-STAT1^{Y701} antibody (Cell Signaling).

### Example 6

### Real-Time RT-PCR

Total RNA was isolated using the RNeasy Mini Kit (Qiagene, Valencia, Calif.) and poly-dT-primed cDNA was synthesized from 3 μg of RNA using Omniscript RT kit (Qiagene). Real-time RT-PCRs were carried out as described in detail elsewhere [9], using Dynamo SYBR Green qPCR chemistry (Finnzymes, Espoo, Finland). The PCR primers were the following (5′ to 3′; product size): CCL3 (SEQ. ID. NO.: 1 and SEQ. ID. NO.: 2), 192 bp; CCL4 (SEQ. ID, NO.: 3 and SEQ. ID. NO.: 4), 214 bp; ACTIN (SEQ. ID. NO.: 5 and SEQ. ID. NO.: 6), 245 bp. The results are expressed as fold difference relative to control (2^{ddC}ₜ).

### Example 7

### NF449 Inhibits FGFR3 Signaling in RCS Chondrocytes

RCS chondrocytes is an FGFR3-expressing chondrocytic cell line that represents the best characterized cellular model for FGFR3-related skeletal dysplasias to date [10]. RCS chondrocytes respond to the FGFR3 activation (via exogenous FGF2 addition) with potent growth arrest, loss of the cartilage-like extracellular matrix and marked alteration of cellular shape [9, 11, 12]. The inventors have taken advantage of the growth-inhibitory response to FGF2 to use RCS cell growth-arrest as a reporter for rapid screening chemical compounds for their activity against FGFR3 signaling [7]. The major advantage of this approach is elimination of toxic compounds as a false-positive hits, given by the nature of RCS response to the FGF2 stimulus, i.e. growth arrest.

The inventors used the RCS growth-arrest assay to screen a molecular library consisting of 1120 molecules (Tocris Bioscience, Ellisville, Mo.). Initial screenings performed at 5, 10 and 20 μM scales identified a compound named NF449 as an antagonist of the FGF2/FGFR3 inhibitory effect on RCS proliferation. This effect was confirmed in detailed cell-growth experiments, where 25 μM NF449 caused nearly 70% reversal of the growth-arrest phenotype without significant cell toxicity throughout its active concentration range (8-30 μM; FIG. 1a). This activity of NF449 is remarkable considering the robust nature of the RCS growth-arrest phenotype [7].

The inventors next compared the NF449 effect with that of C-natriuretic peptide (CNP). CNP is recently discovered, potentially therapeutic antagonist of FGFR3 signaling that suppresses the pathological FGFR3 signaling in cartilage via inhibition of one of its intermediates, the ERK MAP kinase pathway [12, 13]. FIG. 1b shows that CNP caused ˜23% rescue of the FGF2-mediated RCS growth-arrest, similar to previous data [12]. This effect was significantly exceeded by NF449, which lead to ˜50% rescue of the growth arrest phenotype. When used together, CNP and NF449 acted cumulatively, rescuing nearly 80% of the growth arrest.

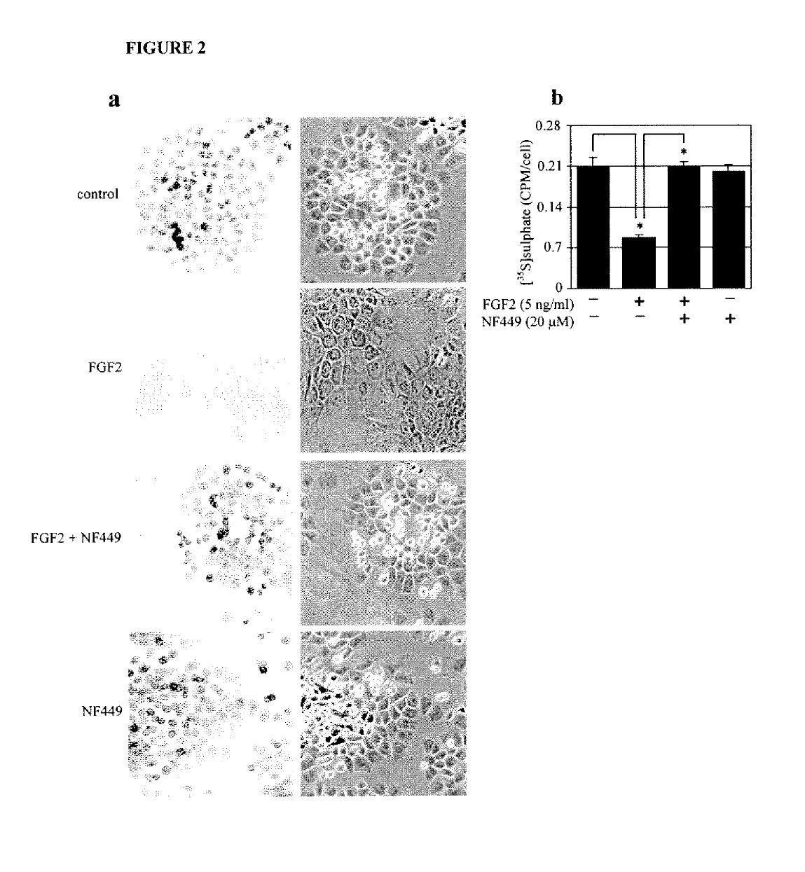

Apart from the growth arrest, aberrant FGFR3 signaling in chondrocytes leads to the loss of their extracellular matrix, rich in sulfated proteoglycans, due to inhibition of matrix synthesis as well as degradation of the existing matrix [12]. The inventors therefore evaluated whether NF449 rescues the extracellular matrix loss induced by FGFR3 activation. Growing RCS cultures produce abundant amounts of sulfated proteoglycan matrix as demonstrated by Alcian blue staining (FIG. 2a). Similar to previous findings [12], this matrix was lost after the 72-hours of FGF2 treatment. In cells treated with FGF2 together with NF449, the intensity of Alcian blue staining equaled that of the control cells, suggesting that NF449 rescues the FGF2-mediated loss of the extracellular matrix. To further confirm this observation, cells were incubated with [³⁵S]sulfate for 72 hours to label the extracellular matrix and determined the effect of FGF2 and NF449 on the cell-bound radioactivity. [³⁵S]sulfate incorporation was significantly inhibited by FGF2 and this effect was completely rescued by NF449 (FIG. 2b). Altogether, the data demonstrates that NF449 potently rescues the FGF2/FGFR3-mediated effects on both chondrocyte proliferation and extracellular matrix loss.

### Example 8

### NF149 Inhibits FGFR3 Signaling in MM Cell Lines OPM2 and KMS11

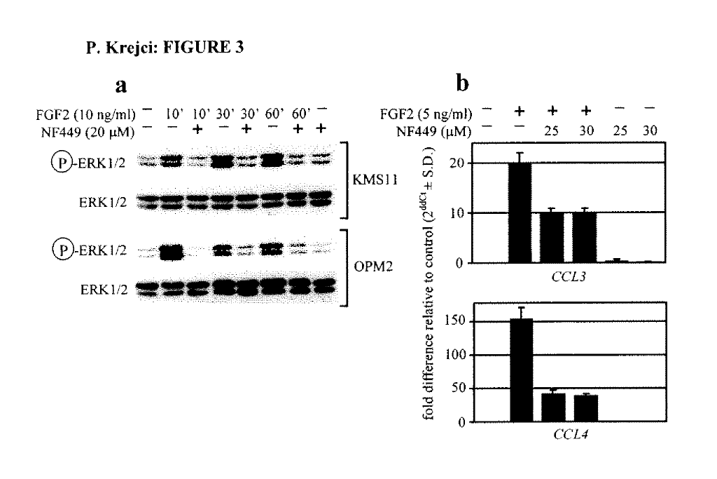

Since MM represents another area of pathological FGFR3 signaling in viva, the inventors evaluated whether NF449 inhibits FGFR3 signaling in KMS11 and OPM2 MM cell lines. OPM2 and KMS11 cells overexpress FGFR3 carrying the strongly activating mutations Y373C or K650E and thus serve as an in vitro model for oncogenic FGFR3 signaling in MM [14]. As a surrogate for FGFR3 activation, ERK MAP kinase was used, which is activated by addition of exogenous FGF2 to the OPM2 or KMS11 cells [15]. FIG. 3a demonstrates that FGF2 stimulation leads to the activatory Thr²⁰²/Tyr²⁰⁴ phosphorylation of ERK, which was almost completely blocked by NF449. To further confirm this data, the effect of NF449 transcription of the genes induced by FGFR3 signaling in MM cells was analyzed. As the CCL3 (MIP-1β) and CCL4 (MIP-1β) chemokines were previously identified as transcriptional targets of FGFR3 signaling in MM [16], the inventors determined the effect of NF449 on the FGF2/FGFR3-induced expression of CCL3 and CCL4. Real-time RT-PCR analysis showed that FGF2-treatment of OPM2 cells leads to significant upregulation of transcripts for both CCL3 and CCL4 that was significantly blocked by NF449 (FIG. 3b).

A little is known about the biological activities of NF449, which was originally described as a G-protein antagonist selective to the G_{SA} subunit [17]. Although NF449 activity against FGFR3 signaling opens the intriguing possibility of involvement of the G proteins, the NF449 concentrations active in the RCS growth-arrest assay were higher than those needed to inhibit [symbol] [17], thus suggesting another target for NF449. The inventors therefore tested the NF449 effect on FGFR3 kinase activity in a cell-free FGFR3 kinase assay that employs recombinant tyrosine kinase domain of FGFR3 as a kinase and STAT1 as a substrate [18]. In the initial experiment, the inventors used a 5-30 μM NF449 concentration range and found FGFR3-mediated tyrosine phosphorylation of STAT1 was inhibited at all concentrations (FIG. 4a). In a broader 0.1-30 μM range, NF449 caused some inhibition of FGFR3 kinase activity at all concentrations although ≧2 μM NF449 was necessary to achieve more than 50% inhibition (FIG. 4b).

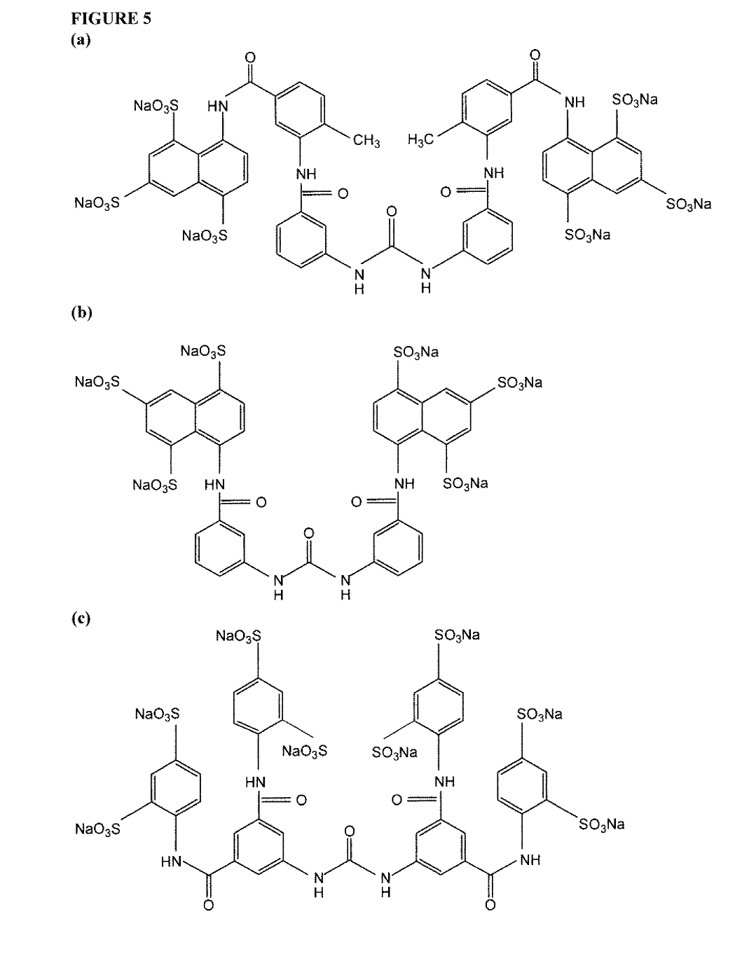

The inventors next tested two other G-protein inhibitors structurally similar to NF449, i.e. suramin and NF023, for their activity against FGFR3. Suramin inhibited FGFR3 kinase activity in contrast to NF023, which had no effect (FIG. 4c, upper blot). These data correspond to the activity of suramin and NF023 in the RCS growth-arrest assay, where suramin reversed the FGF2/FGFR3-mediated growth arrest in a manner similar to NF449 in contrast to NF023 which had no effect (FIG. 4c, lower graph). FIG. 5 shows the chemical structures of suramin, NF449 and NF023.

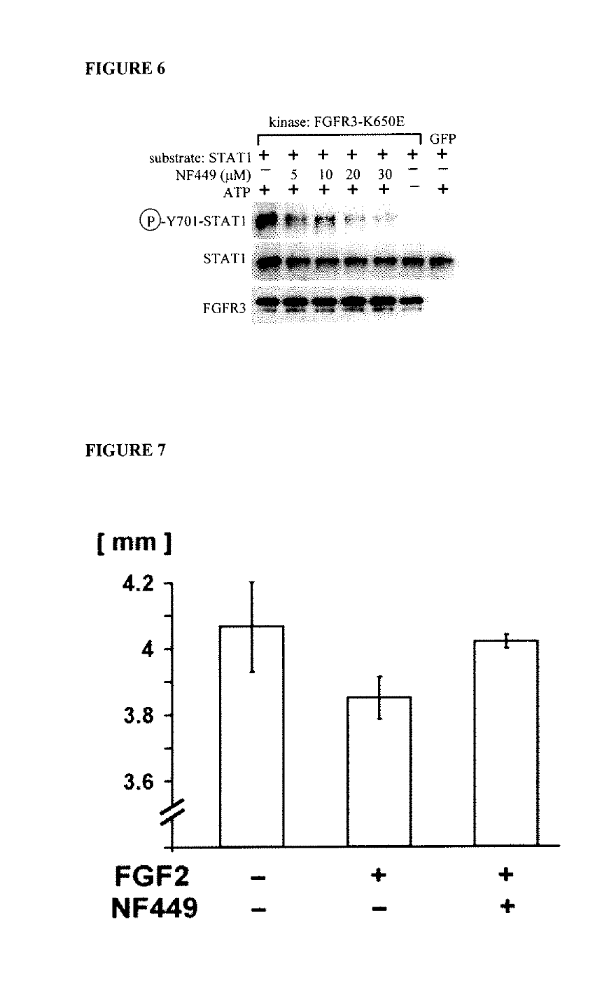

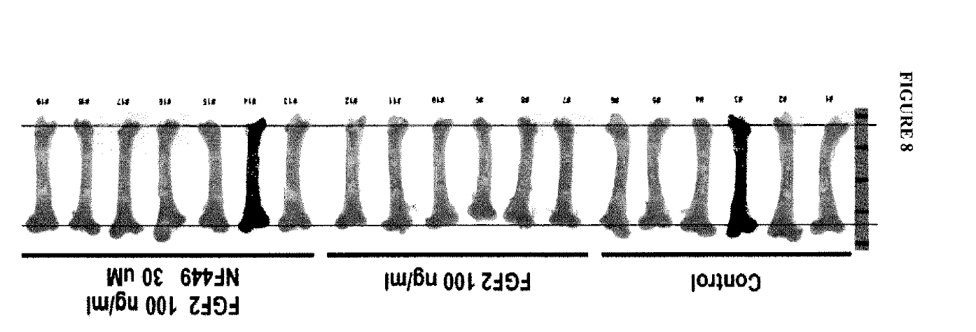

Since OPM2 cells express FGFR3 with a highly activating K650E mutation [14], the inventors used the cell-free kinase assay to determine to what extent the K650E-FGFR3 is inhibited by NF449. As previously described [8], FLAG-tagged full-length FGFR3-K650E was expressed in CHO cells for 48 hours, activated by brief FGF2 treatment and purified by immunoprecipitation. Immunocomplexes were subjected to a kinase assay with STAT1 as a substrate and NF449 added directly to the kinase reaction. FIG. 6 demonstrates that NF449 also inhibited K650E-FGFR3 kinase activity.

### Example 9

### Discussion

The pathological role of FGFR3 receptor tyrosine kinase in several human disorders such as skeletal dysplasias and MM makes FGFR3 an attractive target for therapy. As described herein, the inventors used the growth-inhibitory response of RCS chondrocytes to the FGFR3 activation as a reporter for compound library screening aimed on identification of novel inhibitors of FGFR3 signaling [7]. Using RCS growth-arrest assay, the inventors screened a molecular library consisting of 1120 molecules, leading to identification of several antagonists of FGFR3 signaling.

NF449 inhibited several attributes of FGFR3 signaling in both RCS chondrocytes and MM cell lines OPM2 and KMS11, which represent established in vitro models for FGFR3 signaling in skeletal dysplasias and MM, respectively. These included the rescue of FGF2/FGFR3-induced growth-arrest and extracellular matrix-loss in RCS chondrocytes as well as the inhibition of FGF2/FGFR3-mediated activation of ERK MAP kinase and transcriptional induction of CCL3 and CCL4 chemokines in OPM2 and KMS11 cells, respectively (FIGS. 1-3). As demonstrated by cell-free kinase assays utilizing both recombinant and immunopurified FGFR3 as a kinase and recombinant STAT1 as a substrate [18], NF449 may target FGFR3 signaling in cells by direct inhibition of FGFR3 kinase activity (FIGS. 4, 6).

Remarkably, NF449 rescued the FGF2/FGFR3-mediated growth arrest in RCS chondrocytes more potently that CNP (FIG. 1b), which is considered to be a potential treatment for the FGFR3-related skeletal disorders [13]. Previously, SU5402 was used, which is a well established specific inhibitor of FGFR kinases [19], to inhibit both FGF2-mediated RCS growth arrest and FGFR3 kinase activity. The effects of NF449 in both assays are fully comparable with SU5402, i.e. both drugs inhibit RCS growth arrest and FGFR3 kinase activity in a similar concentration range, 2-20 μM [9, 12] (FIGS. 1a, 4a, 4b, 6).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

[1] Passos-Bueno M R, Wilcox W R, Jabs E W, Sertie A L, Alonso L G, Kitoh H. Clinical spectrum of fibroblast growth factor receptor mutations. Hum Mutat 1999; 14:115-25.
[2] Chesi M, Nardini E, Brents L A, Schrock E, Ried T, Kuehl W M, Bergsagel P L. Frequent translocation t(4;14)(p16.3;q32.3) in multiple myeloma is associated with increased expression and activating mutations in fibroblast growth factor receptor 3. Nat Genet 1997; 16:260-4.
[3] Intini D, Baldini L, Fabris S, Lombardi L, Ciceri G, Maiolo A T, Neri A. Analysis of FGFR3 gene mutations in multiple myeloma patients with t(4;14). Br J Haematol 2001; 114:362-4.
[4] Cappellen D, De Oliveira C, Ricol D et al. Frequent activating mutations of FGFR3 in human bladder and cervix carcinomas. Nat Genet 1999; 23; 18-20.
[5] L'Hote C, Knowles M. Cell responses to FGFR3 signaling: growth, differentiation and apoptosis. Exp Cell Res 2005; 304:417-31.
[6] Plowright E E, Li Z, Bergsagel P L, Chesi M, Barber D L, Branch D R, et al. Ectopic expression of fibroblast growth factor receptor 3 promotes myeloma cell proliferation and prevents apoptosis. Blood 2001; 95:992-8.
[7] Krejci P, Pejchalova K, Wilcox W R. Simple, mammalian cell-based assay for identification of inhibitors of the Erk MAP kinase pathway. Invest New Drugs 2007; 25:391-5.
[8] Krejci P, Masri B, Salazar L, Farrington-Rock C, Prats H, Thompson L M, Wilcox W R. Bisindolylmaleimide I suppresses fibroblast growth factor-mediated activation of Erk MAP kinase in chondrocytes by preventing Shpt association with the Frs2 and Gab1 adaptor proteins. J Biol Chem 2007; 282:2929-36.
[9] Krejci P, Bryja V, Pachernik J, Hampl A, Pogue R, Mekikian B, Wilcox W R. FGF2 inhibits proliferation and alters the cartilage-like phenotype of RCS cells. Exp Cell Res 2004; 297:152-64.
[10] Dailey L, Laplantine E, Priore R, Basilico C. A network of transcriptional and signaling events is activated by FGF to induce chondrocyte growth arrest and differentiation. J Cell Biol 2003; 161:1053-66.
[11] Aikawa T, Sagre G V, Lee K. Fibroblast growth factor inhibits chondrocytic growth through induction of p21 and subsequent inactivation of cyclin E-Cdk2. J Biol Chem 2001; 276:29347-52.
[12] Krejci P, Masri B, Fontaine V, Mekikian P B, Weis M, Prats H, Wilcox W R. Interaction of fibroblast growth factor and C-natriuretic peptide signaling in regulation of chondrocyte proliferation and cartilage matrix homeostasis. J Cell Sci 2005; 118:5089-100.
[13] Yasoda A, Komatsu Y, Chusho H, Miyazawa T, Ozasa A, Miura M, et al. Overexpression of CNP in chondrocytes rescues achondroplasia through a MAPK-dependent pathway. Nat Med 2004; 10:80-6.
[14] Ronchetti D, Greco A, Compasso S, Colombo G, Dell'Era P, Otsuki T, et al. Deregulated FGFR3 mutants in multiple myeloma cell lines with t(4;14): comparative analysis of Y373C, K650E and the novel G384D mutations. Oncogene 2001; 20:3553-62.
[15] Krejci P, Mekikian P B, Wilcox W R. The fibroblast growth factors in multiple myeloma. Leukemia 2006; 20:1165-8.
[16] Masih-Khan E, Trudel S, Heise C, Li Z, Paterson J, Nadeem V, et al. MIP-1□ (CCL3) is a downstream target of FGFR3 and RAS-MAPK signaling in multiple myeloma. Blood 2006; 108:3465-71.
[17] Hohenegger M, Waldhoer M, Beindl W, Böing B, Kreimeyer A, Nickel P, Nanoff C. G_{S□}-selective G protein antagonists. Proc Natl Acad Sci USA 1998; 95:346-51.
[18] Krejci P, Salazar L, Goodridge H S, Kashiwada T A, Schibler M J, Jelinkova P, et al. STAT1 and STAT3 do not participate in FGF-mediated growth arrest in chondrocytes. J Cell Sci 2008; 121:272-81.
[19] Mohammadi M, McMahon G, Sun L, Tang C, Hirth P, Yeh B K, et al. Structures of the tyrosine kinase domain of fibroblast growth factor receptor in complex with inhibitors. Science 1997; 276:955-60.
[20] Kathir K M, Kumar T K, Yu C. Understanding the mechanism of the antimitogenic activity of suramin. Biochemistry 2006; 45:899-906.

1. A method of inhibiting fibroblast growth factor 2 (FGF2) and/or fibroblast growth factor receptor 3 (FGFR3) mediated signaling in a mammal, comprising:
providing a quantity of composition comprising a compound the formula: 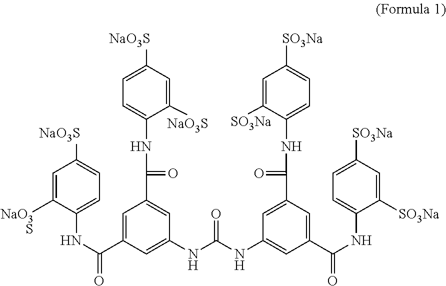
or a pharmaceutical equivalent, analog and/or salt thereof; and
administering the quantity of the composition to the mammal.

2. The method of claim 1, wherein the composition comprises 5 to 30 μM of the compound of Formula 1 or the pharmaceutical equivalent, analog and/or salt thereof.

3. The method of claim 1, wherein the composition comprises greater than 0.1 μM of the compound of Formula 1 or the pharmaceutical equivalent, analog and/or salt thereof.

4. The method of claim 1, wherein the composition comprises at least 2 μM of the compound of Formula 1 or the pharmaceutical equivalent, analog and/or salt thereof.

5. The method of claim 1, wherein the composition comprises 25 μM of the compound of Formula 1 or the pharmaceutical equivalent, analog and/or salt thereof.

6. The method of claim 1, wherein inhibiting FGF2 and/or FGFR3 mediated signaling results in rescue of growth arrest and/or extracellular matrix loss.

7. A method of treating an FGF2 and/or FGFR3 mediated disorder in a subject, comprising:
providing a quantity of a composition comprising a compound of the formula: 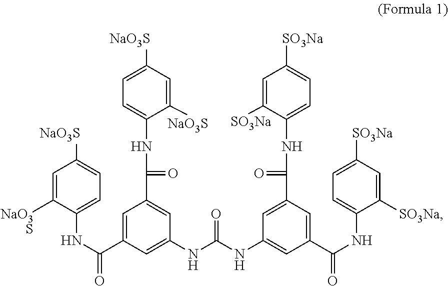
or a pharmaceutical equivalent, analog and/or salt thereof; and
administering the quantity of the composition to the subject.

8. The method of claim 7, wherein the FGF2 and/or FGFR3 mediated disorder comprises a skeletal disorder, skeletal dysplasia, multiple myeloma, cervical carcinoma, and/or bladder carcinoma.

9. The method of claim 7, wherein the composition comprises 25 μM of the compound of Formula 1, or the pharmaceutical equivalent, analog and/or salt thereof.

10. The method of claim 7, wherein the composition comprises from 1 μM to 30 μM of the compound of Formula 1, or the pharmaceutical equivalent, analog and/or salt thereof.

11. The method of claim 7, wherein the composition is administered to the subject intravenously.

12. The method of claim 7, wherein the composition is administered to the subject by direct injection.

13. The method of claim 7, wherein FGF2 and/or FGFR3 mediated signaling is inhibited by direct inhibition of FGFR3 kinase activity.

14. A pharmaceutical composition, comprising:
a therapeutically effective amount of a compound of the formula: 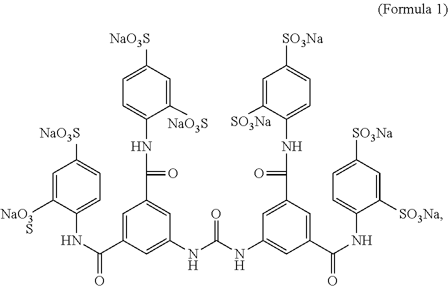
or a pharmaceutical equivalent, analog and/or salt thereof; and
a pharmaceutically acceptable carrier.

15. A method of treating an FGF2 and/or FGFR3 mediated condition in a subject, comprising:
administering a quantity of a composition comprising a compound of the formula: 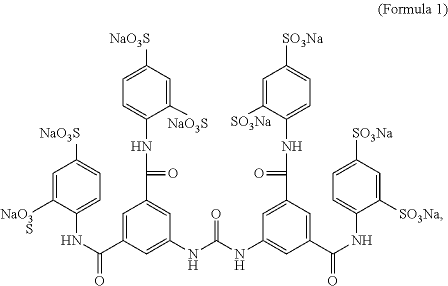
or a pharmaceutical equivalent, analog and/or salt thereof, to the subject; and
administering a quantity of a composition comprising a C-natriuretic peptide (CNP) compound, or a pharmaceutical equivalent, analog, derivative and/or salt thereof, to the subject.


SEQUENCE LISTING (6 sequences)

SEQ ID NO: 1 - DNA, 21 bp, Homo sapiens
cacattccgtcacctgctcag

SEQ ID NO: 2 - DNA, 23 bp, Homo sapiens
tggctgctcgtctcaaagtagtc

SEQ ID NO: 3 - DNA, 21 bp, Homo sapiens
tctcagcaccaatgggctcag

SEQ ID NO: 4 - DNA, 24 bp, Homo sapiens
cagttcagttccaggtcatacacg

SEQ ID NO: 5 - DNA, 21 bp, Homo sapiens
gattcctatgtgggcgacgag

SEQ ID NO: 6 - DNA, 23 bp, Homo sapiens
atggctggggtgttgaaggtctc